(19) United States
(12) Patent Application Publication
(10) Pub. No.: US 2004/0073269 A1
(43) Pub. Date: Apr. 15, 2004

(54) Combined percutaneous/surface electrical stimulation

(76) Inventors: William J. Carroll, Sedro Woolley, WA (US); Richard M. Terrell, Vancouver, WA (US)

Correspondence Address: BLANK ROME LLP, 600 NEW HAMPSHIRE AVENUE, N.W., WASHINGTON, DC, 20037, US

(21) Appl. No.: 10/652,562

(22) Filed: Sep. 2, 2003

Related U.S. Application Data
(60) Provisional application No. 60/406,914, filed on Aug. 30, 2002.

(51) Int. Cl. A61N 1/18

(52) U.S. Cl. 607/46

(57) ABSTRACT

A stimulator and a method for integrated electrical stimulation of muscle, nerves and tissues. A combined percutaneous/transcutaneous stimulator using integrated surface electrodes delivers electric pulses simultaneously to deep tissue and a skin surface of a target area. The integrated surface electrodes include fine needle electrodes integrated with surface electrodes. The combined percutaneous/transectaneous stimulator with the integrated surface electrodes affords quick pain relief, more comfortable needle electrode insertion and longer lasting pain reduction results.

### REFERENCE TO RELATED APPLICATION

The present application claims the benefit of U.S. Provisional Patent Application No. 60/406,914, filed Aug. 30, 2002, whose disclosure is hereby incorporated by reference in its entirety into the present disclosure.

### FIELD OF THE INVENTION

The present invention is generally related to electrical stimulation, and, more particularly, to an electro-medical device and method for integrated percutaneous and transcutaneous electrical stimulation of muscle, nerves and tissues.

### BACKGROUND OF THE INVENTION

Percutaneous electrical stimulation (also referred to as Percutaneous neuromodulation therapy or (PNT) delivers electrical pulses directly to the deep tissues by means of but not limited to fine needle electrodes that are inserted preferably to a dept of approximately 2-3 centimeters. Percutaneous, as defined, is through the skin and usually utilizes fine wire electrodes that stimulate deep musculature and nerves along with other tissues. Percutaneous neuromodulation therapy (PNT) does not always show a positive effect immediately after treatment and may take up to 4 treatment sessions before any benefit is perceived. Seroussi RE, et al., “Effectiveness of Percutaneous Neuromodulation Therapy for Patients with Chronic and Severe Low Back Pain.” Accepted for publication, Pain Practice, Volume 3, Issue Mar. 1, 2003. The advantage of percutaneous neuromodulation therapy is that it seems to provide longer lasting pain relief of 6 months or more with a series of 10 sessions. Ghoname E A, et al. Percutaneous electrical nerve stimulation for low back pain: a randomized crossover study. JAMA 1999;281:818-23.

Transcutaneous electrical stimulation is defined as passing various types of current across the skin using electrodes that are placed on the surface of the skin. This type of surface stimulation has been shown to provide relatively rapid onset of pain control, Robinson A J, et al. Clinical Electrophysiology, Second Edition. Baltimore: Williams & Wilkins, 1995: 285-290. But the relief from surface stimulation customarily is of short duration, (i.e., hours or at best days) Johnson M I, et al. An In-Depth Study of Long-Term Users of Transcutaneous electrical Nerve Stimulation (TENS). Implications for clinical use of TENS. PAIN 1991; 41: 221-229. Hans J S, et al. Effect of low- and high-frequency TENS on Met-enkephalin-Arg-Phe and dynorphin A immunoreactivity in human lumbar CSF. PAID 1991; 47: 295-298. Examples of this type of stimulation are but should not be limited to, Transcutaneous Electrical Nerve Stimulation (TENS), NeroMuscular Electrical Stimulation (NMES), Interferential Stimulation, Diadynamic Stimulation, High Volt Galvanic Stimulation (HVGS), Electro-Magnetic and Pulsed Electro-Magnetic Field Stimulation (EMF & PEMF) and Micro-current Stimulation. Nelson R M, Clinical Electrotherapy, Third Edition. Stamford: Appleton & Lange, 1999: 316-319. Transcutaneous electrical stimulation can be useful for many pain conditions but is limited in its duration of effect and is palliative at best. Combining it with a longer lasting re-modulating technique such as PNT would produce a more comfortable and effective result. Patients would also begin to see results and benefits much sooner than PNT treatment alone.

However, as described above, using percutaneous stimulation does not necessarily show a positive effect immediately after treatment and may take up to four treatment sessions before any benefit is perceived. Transcutaneous stimulation, on the other hand, is customarily of short duration of effect but, has a rapid onset of control. Also, the fine needle electrodes used in percutaneous stimulation are quite uncomfortable when inserted.

Thus, a heretofore unaddressed need exists in the industry to address the aforementioned deficiencies and inadequacies.

### SUMMARY OF THE INVENTION

This invention is primarily directed for use in a medical clinic environment for the treatment of both radicular and axial components of Low Back Pain, Cervical Pain and other Pain Syndromes.

Embodiments of the present invention combine surface stimulation with percutaneous electrical stimulation (see FIG. 1). In one embodiment, the system has electrodes and circuits that are part of the needle positioning system that provide stimulation to superficial afferent nerves and provide quick analgesia to the patient. Fast onset of pain relief could be a benefit for needle electrode insertion and patient compliance. The surface stimulation system is integrated into the PNT system.

In another embodiment, both the transcutaneous and percutaneous stimulation systems are separate. However, the transcutaneous stimulation system is used in conjunction with the percutaneous system (see FIG. 2).

The benefit of linking the use of surface stimulation with percutaneous is that the patient achieves quick relief of pain, more comfortable needle electrode insertion and ultimately, longer lasting results.

### BRIEF DESCRIPTION OF THE DRAWINGS

Many aspects of the invention can be better understood with reference to the following drawings. The components in the drawings are not necessarily to scale, emphasis instead being placed clearly upon illustrating the principles of the present invention. Moreover, in the drawings, like reference numerals designate corresponding parts throughout the several views.

FIG. 1 is a perspective view illustrating an embodiment of the invention; and

FIG. 2 is a perspective view illustrating another embodiment of the invention.

### DETAILED DESCRIPTION OF THE PREFERRED EMBODIMENT

A preferred embodiment of the invention and modifications thereof will now be described with reference to the drawings.

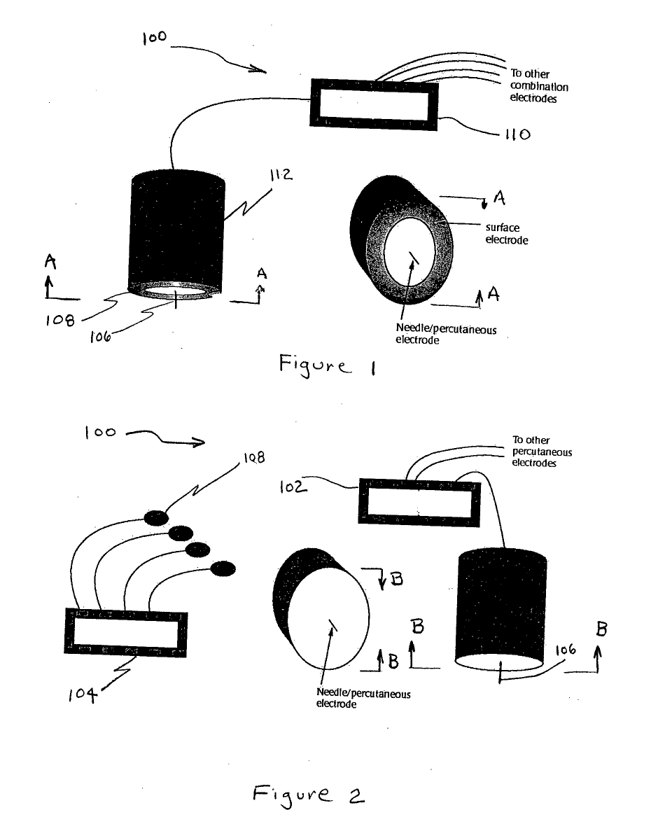

FIG. 1 shows an electro-medical device 100 for electrically stimulating a target area (not shown) of a subject. The target area (not shown) includes both the deep tissues (not shown) and the surface of the skin (not shown). The electro-medical device 100 uses a combined percutaneous/transcutaneous stimulator 110 to simultaneously generate electrical pulses directly to the deep tissues of the target area (not shown), and the surface of the skin of the target area (not shown). Using the combined percutaneous/transcutaneous stimulator 110 to simultaneously generate the electrical pulses for stimulation combines a long duration of pain relief afforded by percutaneous stimulation, with a short duration, coupled to a rapid onset of pain relief afforded with the use of transcutaneous stimulation.

The generated electrical pulses are transmitted to the target area (not shown) by integrated surface electrodes 112. The integrated surface electrodes 112 are a combination of surface electrodes 108 used with a transcutaneous stimulator 104 (FIG. 2), and fine needle electrodes 106 used with a percutaneous stimulator 102 (FIG. 2). By using integrated surface electrodes 112, electrical pulses are delivered both directly to the deep tissues, musculature and nerves (percutaneous stimulation) and transmitted to superficial afferent nerves via the skin surface (transcutaneous stimulation). The combined percutaneous/transcutaneous stimulator 110 using the integrated surface electrodes 112 provides quick relief of pain, more comfortable fine needle electrode insertion and longer lasting pain relief to a patient.

Results similar to those obtained with the percutaneous/transcutaneous stimulator 110 using the integrated surface electrodes 112 are achievable by using a transcutaneous stimulator 104 with surface electrodes 108 in conjunction with a separate percutaneous stimulator 102 using fine needle electrodes 106 (see FIG. 2). However, as mentioned above, the combined percutaneous/transcutaneous stimulator 110 provides the benefits afforded by both stimulation methods in one treatment.

The percutaneous/transcutaneous stimulator 110 could be utilized to generate an interferential current with a base medium frequency of at least 1 KHz but no more than 20 kHz, and a resultant beat frequency of no more than 250 Hz. The interferential current, transmitted transcutaneously could promote osteogenesis, and aid in the treatment of osteoporosis.

We claim:

1. An electro-medical device for electrical stimulation of a target area, comprising:
a percutaneous stimulator; and
a transcutaneous stimulator connected to said percutaneous stimulator.

2. The electro-medical device of claim 1, wherein said percutaneous stimulator comprises fine needle electrodes.

3. The electro-medical device of claim 1, wherein said transcutaneous stimulator comprises surface electrodes.

4. The electo-medical device of claim 1, wherein said percutaneous stimulator delivers electrical pulses directly to deep tissues.

5. The electro-medical device of claim 1, wherein said transcutaneous stimulator delivers electrical pulses to a surface of the target area.

6. The electro-medical device, of claim 1, wherein said percutaneous stimulator achieves a long duration of pain relief.

7. The electro-medical device of claim 1, wherein said transcutaneous stimulator achieves a short duration of pain relief.

8. The electro-medical device of claim 1, wherein said transcutaneous stimulator achieves rapid pain relief.

9. The electro-medical device of claim 1, wherein said transcutaneous stimulator generates an interferential current with a base medium frequency of at least 1 kHz but no more than 20 KHz.

10. The electro-medical device of claim 9, wherein the interferential current includes a resultant beat frequency of no more than 0-250 Hz.

11. An electro-medical device for integrated electrical stimulation of a target area comprising a combined percutaneous/transcutaneous stimulator.

12. The electro-medical device of claim 11, wherein said transcutaneous stimulator comprises integrated surface electrodes.

13. The integrated surface electrodes of claim 12, further comprising fine needle electrodes combined with surface electrodes.

14. The electro-medical device of claim 11, wherein said combined percutaneous/transcutaneous stimulator delivers electrical pulses directly to deep tissues.

15. The electro-medical device of claim 11, wherein said combined percutaneous/transcutaneous stimulator delivers electrical pulses to a surface of the target area.

16. The electro-medical device of claim 11, wherein said combined percutaneous/transcutaneous stimulator delivers electrical pulses simultaneously to deep tissues and a surface of the target area.

17. The electro-medical device of claim 11, wherein said combined percutaneous/transcutaneous stimulator achieves a long duration of pain relief.

18. The electro-medical device of claim 11, wherein said combined percutaneous/transcutaneous stimulator achieves rapid pain relief.

19. The electro-medical device of claim 11, wherein said combined percutaneous/transcutaneous stimulator generates an interferential current with a base medium frequency of at least 1 kHz but no more than 20 KHz.

20. The electro-medical device of claim 20, wherein the interferential current includes a resultant beat frequency of 0-250 Hz.

21. A means for simultaneous electrical stimulation of deep tissues and a surface of a target area using an electrical stimulator.

22. The means according to claim 22, wherein the electrical stimulator is a combined percutaneous/transcutaneous stimulator.

23. The means according to claim 23, wherein said combined perceutaneous/transculaneous stimulator includes integrated surface electrodes.

24. The means according to claim 24, wherein said integrated surface electrodes comprise fine needles electrodes combined with surface electrodes.

25. The means according to claim 22, wherein said means achieves a long duration of pain relief.

26. The means according to claim 22, wherein the means achieves rapid pain relief.

27. The means according to claim 22, wherein the means generates an interferential current with a base medium frequency of at least 1 kHz but no more than 20 KHz.

28. The means according to claim 27, wherein the interferential current includes a resultant beat frequency of 0-250 Hz.

29. A method for electrically stimulating a target area using a combined percutaneous/transcutaneous stimulator.

30. The method according to claim 29, said method comprising using integrated surface electrodes.

31. The method according to claim 30, wherein said integrated surface electrodes comprise fine needle electrodes combined with surface electrodes.

32. The method according to claim 29, said method comprising delivering electrical pulses directly to deep tissues.

33. The method according to claim 29, said method comprising delivering electrical pulses to a surface of the target area.

34. The method according to claim 29, said method comprising delivering electrical pulses simultaneously to deep tissues and a surface of the target area.

35. The method according to claim 29, said method achieving a long duration of pain relief.

36. The method according to claim 29, said method achieving rapid pain relief.

37. The method according to claim 29, said method generating an interferential current with a base medium frequency of at least 1 KHz but no more than 20 KHz.

38. The method according to claim 37, said method further comprising generating the inferential current with a resultant beat frequency of 0-250 Hz.
